(19) United States
(12) Patent Application Publication
(10) Pub. No.: US 2019/0343374 A1
(43) Pub. Date: Nov. 14, 2019

(54) ENDOSCOPE

(71) Applicant: FUJIFILM Corporation, Tokyo (JP)

(72) Inventors: Hiroshi SHIBUYA, Ashigarakami-gun (JP); Kenji YAMAKAWA, Ashigarakami-gun (JP)

(73) Assignee: FUJIFILM Corporation, Tokyo (JP)

(21) Appl. No.: 16/404,757

(22) Filed: May 7, 2019

(30) Foreign Application Priority Data
May 9, 2018 (JP) 2018-090697

(51) Int. Cl. A61B 1/00; A61B 1/06; G02B 23/24; A61B 1/015; A61B 1/12

(52) U.S. Cl. CPC A61B 1/00165; A61B 1/0676; G02B 23/2469; A61B 1/00135; A61B 1/05; A61B 1/12; G02B 23/2423; A61B 1/00091; A61B 1/015

(57) ABSTRACT

Provided is an endoscope that can suppress breakage of an optical fiber and has excellent assemblibility and handlability. An endoscope includes a light guide that guides illumination light created by a light source device from the light source device via an operating part of the endoscope to a distal end part of an insertion part of the endoscope through an interior of an endoscope. The light guide includes a first optical fiber bundle; a first protective tube that covers the first optical fiber bundle from the distal end part to the operating part; and a second protective tube that is connected to a proximal end part of the first protective tube and covers the first optical fiber bundle. The first protective tube is made of a non-porous (solid) fluororesin. The second protective tube has a bending stiffness smaller than the first protective tube.

### CROSS-REFERENCE TO RELATED APPLICATIONS

This application is based on and claims priority under 35 USC 119 from Japanese Patent Application No. 2018-090697 filed on May 9, 2018, the contents of which are incorporated herein by reference.

### BACKGROUND OF THE INVENTION

### 1. Field of the Invention

The present invention relates to an endoscope.

### 2. Description of the Related Art

Illumination light of endoscopes is created by a light source device and is supplied from the light source device to the endoscopes. The endoscopes comprise an insertion part to be inserted into a subject, an operating part connected to the insertion part, and a universal cord extending from the operating part to the light source device, and a light guide including an optical fiber bundle is inserted through interiors of the universal cord, the operating part, and the insertion part. The illumination light supplied to the endoscopes is guided from the terminal of the universal cord to a distal end part of the insertion part through the light guide. In order to suppress breakage of an optical fiber, the optical fiber bundle is typically covered with a protective tube. As materials of the protective tube, for example, polytetrafluoroethylene (PTFE), silicone resin, urethane resin, and the like are used (for example, refer to JP1995-181397A (JP-H07-181397A)).

Additionally, a light guide in which the optical fiber bundle is covered with different protective tubes for different regions is known. For example, in a light guide described in JP1985-176015A (JP-S60-176015A), a portion ranging from a distal end part of an insertion part to a bending part is covered with a protective tube having a two-layer structure of an inner layer made of stretched foamable tetrafluoroethylene and an outer layer made of fluororubber, and a portion closer to a proximal end side than the bending part is covered with silicone rubber. Additionally, in a light guide described in JP6257852B, an optical fiber bundle is branched into two on a distal end part side of an insertion part, a portion ranging from the distal end part of the insertion part to a branching part is covered with a protective tube made of stretched porous PTFE, and a portion closer to a proximal end side than the branching part is covered with a protective tube made of silicone resin.

### SUMMARY OF THE INVENTION

A factor of the breakage of the optical fiber includes meandering of the light guide. The light guide is pushed and pulled in a longitudinal direction with bending and bending-releasing operations of the bending part. For example, the light guide pushed from the bending part with the bending operation comes into contact with other built-in objects (for example, an electrical cable to be connected to an imaging element, operating wires for bending the bending part, a treatment tool channel through which a treatment tool is inserted, and the like) inserted through the insertion part.

In a case where the other built-in objects and the light guide come into contact with each other, due to friction, it is difficult to push the light guide and the light guide meanders. In a case where the light guide meanders, for example, the light guide is pushed against the other built-in objects. Accordingly, stress is applied to an optical fiber, and the optical fiber breaks due to this stress.

In order to suppress the breakage of the optical fiber resulting from the meandering of the light guide, it is important to reduce the friction between the light guide and the other built-in objects, and PTFE is suitable among the above enumerated materials of the protective tube. The PTFE containing fluorine generally has a low friction than the silicone resin or the like.

Additionally, from a viewpoint of not hindering the bending operation of the bending part up to now, relatively flexible stretched porous PTFE among PTFEs is used for the material of the protective tube (for example, refer to JP1985-176015A and JP6257852B). However, in order to suppress the breakage of the optical fiber resulting from the meandering of the light guide, it is also important for the protective tube to have moderate bending stiffness. In a case where the protective tube has the moderate bending stiffness, the meandering of the light guide resulting from the bending and bending-releasing operations of the bending part can be suppressed.

However, in a case where the optical fiber bundle is covered with a protective tube of high stiffness over the entire length of the light guide, there is a concern about degradation of the assemblibility and handlability of the endoscope. Thus, as in the light guide described in JP1985-176015A, it is considered that the optical fiber bundle is covered with a high-stiffness protective tube, in the bending part in which the bending and bending-releasing operations are frequently performed, and the optical fiber bundle is covered with a low-stiffness protective tube, in a portion closer to a proximal end side than the bending part. However, in this case, in a flexible part in which the bending stiffness of the protective tube varies, there is a concern that the light guide may meander.

The invention has been made in view of the above-described circumstances, and an object thereof is to provide an endoscope that can suppress breakage of an optical fiber and has excellent assemblibility and handlability.

An endoscope of one aspect of the invention comprises a light guide that guides illumination light generated by a light source device from the light source device via an operating part of the endoscope to a distal end part of an insertion part of the endoscope through an interior of the endoscope. The light guide includes a first optical fiber bundle; a first protective tube that covers the first optical fiber bundle from the distal end part of the insertion part to the operating part; and a second protective tube that is connected to an end part of the first protective tube on a side of the light source device and covers the first optical fiber bundle. The first protective tube is made of a non-porous (solid) fluororesin. The second protective tube has a bending stiffness smaller than a bending stiffness of the first protective tube.

According to the invention, it is possible to provide the endoscope that can suppress breakage of an optical fiber and has excellent assemblibility and handlability.

### BRIEF DESCRIPTION OF THE DRAWINGS

FIG. 1 is a perspective view of an example of an endoscope for explaining an embodiment of the invention.

FIG. 2 is a schematic view of an example of an endoscope system including the endoscope of FIG. 1.

FIG. 3 is a plan view of a distal end surface of an insertion part of the endoscope of FIG. 1.

FIG. 4 is a schematic view of one configuration example of a light guide of the endoscope of FIG. 1.

FIG. 5 is a schematic view of another configuration example of the light guide of the endoscope of FIG. 1.

FIG. 6 is a schematic view of still another configuration example of the light guide of the endoscope of FIG. 1.

### DESCRIPTION OF THE PREFERRED EMBODIMENTS

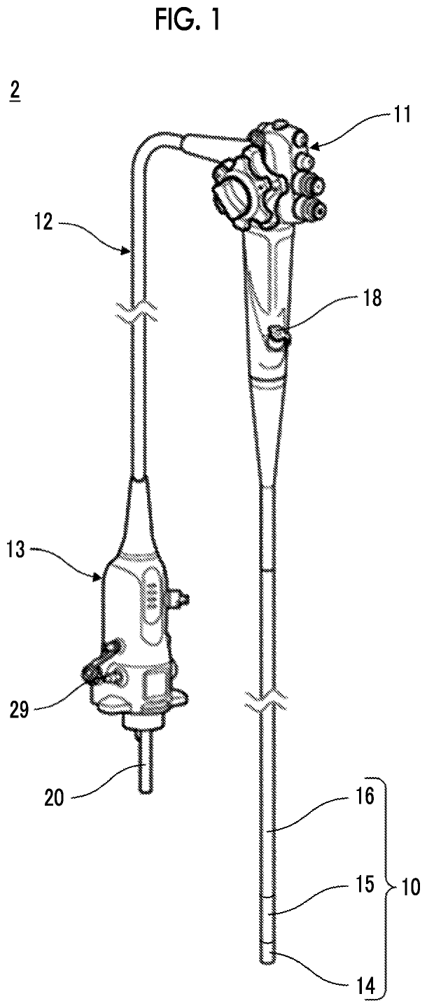

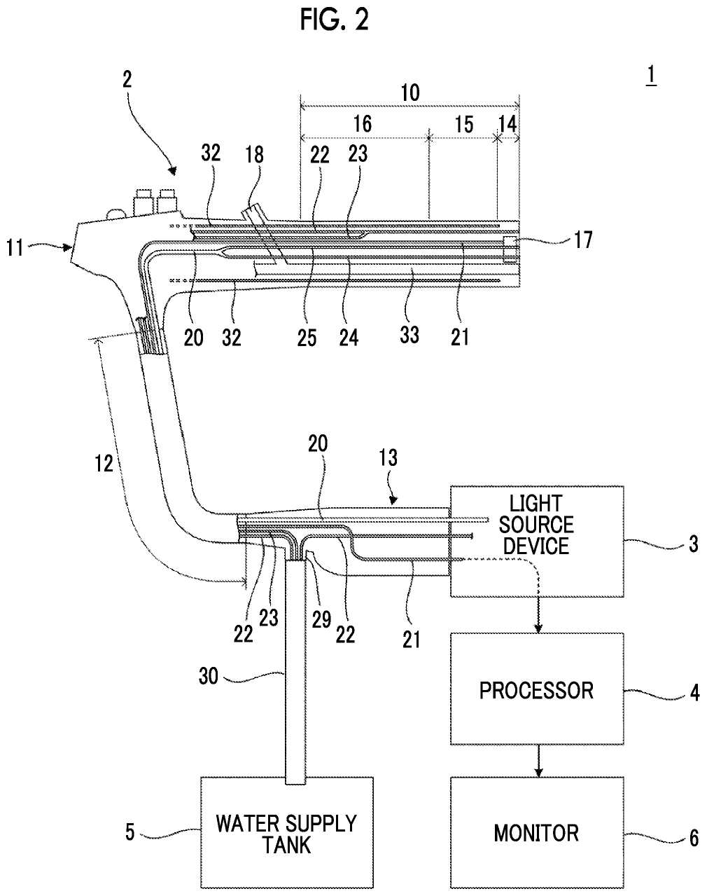

FIG. 1 illustrates an example of an endoscope for explaining an embodiment of the invention, and FIG. 2 illustrates an example of an endoscope system including the endoscope of FIG. 1.

The endoscope system 1 comprises an endoscope 2, a light source device 3, a processor 4, and a water supply tank 5. An endoscope 2 has the insertion part 10 inserted into a subject, the operating part 11 connected to the insertion part 10, and the universal cord 12 extending from the operating part 11, and a terminal of the universal cord 12 is provided with a connector 13 to be connected to the light source device 3.

The insertion part 10 is constituted of a distal end part 14, a bending part 15 connected to the distal end part 14, and a flexible part 16 to which the bending part 15 and the operating part 11 are connected. An imaging device 17 including imaging elements, such as a charge coupled device (CCD) image sensor and a complementary metal oxide semiconductor (CMOS) image sensor, is mounted on the distal end part 14. The bending part 15 is configured to be bendable, and bending of the bending part 15 is operated by the operating part 11. Additionally, the flexible part 16 is configured to be flexible so as to be deformable along the shape of an insertion path of the subject.

The operating part 11 is provided with an operation button that operates imaging using the imaging device 17, an operation button that operates air supply and/or water supply for sending gas and/or liquid to the distal end part 14, an operation knob that operates the bending of the bending part 15, and the like. Additionally, the operating part 11 is provided with a treatment tool insertion port 18 into which treatment tools, such as forceps, can be inserted.

A light guide 20, an electrical cable 21, an air supply pipe 22, and a water supply pipe 23 are provided inside the insertion part 10, the operating part 11, and the universal cord 12.

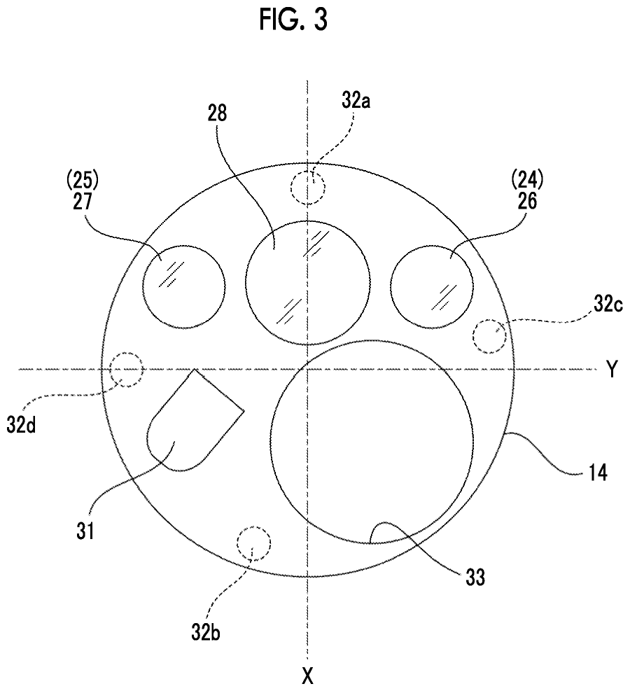

The light guide 20 guides illumination light, which is to be created by the light source device 3, to the distal end part 14. As illustrated in FIGS. 2 and 3, the light guide 20 has a first optical fiber bundle 24 and a second optical fiber bundle 25. The first optical fiber bundle 24 and the second optical fiber bundle 25 are separated from each other from the distal end part 14 to the operating part 11 and are joined to each other inside the operating part 11. An end surface of the distal end part 14 is provided with a first illumination window 26 for emitting the illumination light guided by the first optical fiber bundle 24 and a second illumination window 27 for emitting the illumination light guided by the second optical fiber bundle 25. The first illumination window 26 and the second illumination window 27 are disposed with an observation window 28 of the imaging device 17 interposed therebetween, and thereby, illumination unevenness is reduced.

The electrical cable 21 transmits operating power, control signals, and image signals of the imaging device 17 between the imaging device 17 and the processor 4. The processor 4 processes the input image signals to create image data of an observation region within the subject, displays the created image data on a monitor 6, and records the created image data.

The air supply pipe 22 sends gas (for example, air), which is to be used for cleaning or the like of the observation window 28, to the distal end part 14, and the water supply pipe 23 sends liquid (for example, water), which is to be used for cleaning or the like of the observation window 28, to the distal end part 14. The connector 13 is provided with a mouthpiece 29, and the air supply pipe 22 and the water supply pipe 23 are connected to the water supply tank 5 via a connecting pipe 30 to be connected to a mouthpiece 29. As illustrated in FIGS. 2 and 3, the air supply pipe 22 and the water supply pipe 23 are joined together in the flexible part 16 and reaches the distal end part 14. The end surface of the distal end part 14 is provided with a nozzle 31 that sprays the gas sent by the air supply pipe 22 and the liquid sent by the water supply pipe 23 toward the observation window 28.

Additionally, a plurality of operating wires 32, and a treatment tool channel 33 are provided inside the insertion part 10 and the operating part 11.

The operating wires 32 reach the operating part 11 from the distal end part 14 and are pushed toward the distal end part 14 side or pulled toward the operating part 11 side depending on the operation of the above operation knob of the operating part 11. As illustrated in FIG. 3, the bending part 15 is bendable in an upward-downward direction along a first axis X orthogonal to a longitudinal axis of the insertion part 10 and in a leftward-rightward direction along a second axis Y orthogonal to the longitudinal axis and the first axis X. The plurality of operating wires 32 includes an operating wire 32a and an operating wire 32b that are disposed with the longitudinal axis of the insertion part 10 interposed therebetween in the upward-downward direction, and an operating wire 32c and an operating wire 32d that are disposed with the longitudinal axis of the insertion part 10 interposed therebetween in the leftward-rightward direction. As one operating wire of the operating wire 32a and the operating wire 32b is pushed toward the distal end part 14 side and the operating wire thereof is pulled toward the operating part 11 side, the bending part 15 is bent upward or downward. Similarly, as one operating wire of the operating wire 32c and the operating wire 32d is pushed toward the distal end part 14 side and the operating wire thereof is pulled toward the operating part 11 side, the bending part 15 is bent leftward or rightward.

The treatment tool channel 33 reaches the distal end part 14 from the treatment tool insertion port 18 of the operating part 11 and opens to the end surface of the distal end part 14. A treatment tool inserted into the treatment tool insertion port 18 is guided by the treatment tool channel 33 and is protruded from an opening of the treatment tool channel 33 in the end surface of the distal end part 14.

As illustrated in FIG. 3, the first illumination window 26 for emitting the illumination light guided by the first optical fiber bundle 24 and the second illumination window 27 for emitting the illumination light guided by the second optical fiber bundle 25, and the observation window 28 of the imaging device 17 are disposed on one side above or below the second axis Y and disposed side by side in the leftward-rightward direction along the second axis Y, due to a relationship of arrangement of other built-in objects, such as the treatment tool channel 33. The first optical fiber bundle 24 is disposed in the vicinity of one operating wire 32c of the operating wire 32c and the operating wire 32d that are disposed with the longitudinal axis of the insertion part 10 interposed therebetween in the leftward-rightward direction, and the second optical fiber bundle 25 is disposed in the vicinity of the other operating wire 32d. While the operating wire 32d is disposed on the second axis Y, the operating wire 32c is disposed so as to be biased to the same side as the illumination window 26, the illumination window 27, and the observation window 28 with respect to the second axis Y. For this reason, the spacing between the first optical fiber bundle 24 and the operating wire 32c is narrower than the spacing between the second optical fiber bundle 25 and the operating wire 32d, and the other built-in objects are disposed relatively densely around the first optical fiber bundle 24.

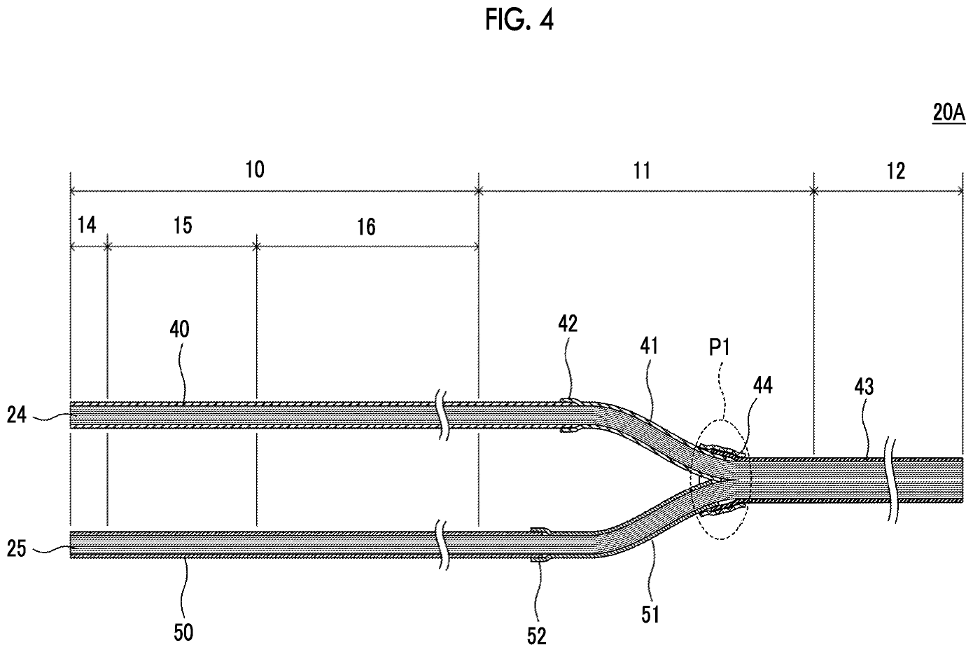

FIG. 4 illustrates a configuration example of the light guide 20.

The light guide 20A illustrated in FIG. 4 has the first optical fiber bundle 24 and the second optical fiber bundle 25 as described above, and the first optical fiber bundle 24 and the second optical fiber bundle 25 are separated from each other from the distal end part 14 to the operating part 11 and are joined to each other inside the operating part 11. The light guide 20 has a first protective tube 40, a second protective tube 41, and a third protective tube 43, as protective tubes that cover the first optical fiber bundle 24.

The first protective tube 40 covers the first optical fiber bundle 24 from the distal end part 14 to the operating part 11. The first protective tube 40 is made of a non-porous (solid) fluororesin. The fluororesin is, for example, polytetrafluoroethylene (PTFE), perfluoroalkoxyalkane (PFA), perfluoroethylene propene copolymers (FEP), or the like.

The second protective tube 41 is connected to a proximal end part (an end part on the light source device 3 side) of the first protective tube 40 and covers the first optical fiber bundle 24. The second protective tube 41 has a bending stiffness smaller than the first protective tube 40. Porous fluororesin, polyolefin, silicone resin, and the like can be exemplified as materials of the second protective tube 41. In addition, the bending stiffness is a bending elastic modulus as defined in Japanese Industrial Standards (JIS) K7171 “Method of Determining Plastics-Bending Properties.”

The first optical fiber bundle 24 is bundled with the second optical fiber bundle 25 in a portion covered with the second protective tube 41. That is, the second protective tube 41 covers the first optical fiber bundle 24 from the proximal end part of the first protective tube 40 to a joining point P1 between the first optical fiber bundle 24 and the second optical fiber bundle 25 inside the operating part 11. The first protective tube 40 and the second optical fiber bundle 25 with relatively large bending stiffness do not come into contact with each other, and damage to the second optical fiber bundle 25 is suppressed. In the joining point P1, it is preferable to fix the third protective tube 43 using a binding member 44, such as a heat-shrinkable tube.

The third protective tube 43 is connected to a proximal end part of the second protective tube 41 and integrally covers the first optical fiber bundle 24 and the second optical fiber bundle 25. The third protective tube 43 has a bending stiffness smaller than the second protective tube 41. Silicone resin can be exemplified as a material of the third protective tube 43.

The light guide 20 is pushed and pulled in the longitudinal direction with bending and bending-releasing operations of the bending part 15. For example, the light guide 20 pushed from the bending part 15 with the bending operation comes into contact with the other built-in objects (the electrical cable 21, the air supply pipe 22, the water supply pipe 23, the operating wires 32, the treatment tool channel 33, and the like) in a case where the light guide 20 is pushed. In the fine-diameter insertion part 10, any contact between the built-in objects including light guide 20 is likely to occur.

The first protective tube 40 made of the non-porous (solid) fluororesin has low friction. Even in a case where any contact between the first protective tube 40 and the other built-in objects has occurred in a case where the light guide 20 is pushed, it is suppressed that the first optical fiber bundle 24 covered with the first protective tube 40 deflects more than needed along gaps between the first optical fiber bundle 24 and the other built-in objects. The first protective tube 40 made of the non-porous (solid) fluororesin has high stiffness, and the deflection of the first optical fiber bundle 24 more than needed along the gaps between the first optical fiber bundle 24 and the other built-in objects is suppressed. The deflection more than needed is similarly suppressed also regarding the movement of the light guide 20 resulting from looping or the like of the flexible part 16. Moreover, the first protective tube 40 covers the first optical fiber bundle 24 from the distal end part 14 to the operating part 11, and the bending stiffness of the first optical fiber bundle 24 becomes constant over the entire length of the insertion part 10. Accordingly, meandering of the first optical fiber bundle 24 can be suppressed, and breakage of an optical fiber resulting from the meandering can be suppressed.

It is preferable that a filling factor that is a ratio between the inner cross-sectional area of the first protective tube 40 and the cross-sectional area of the first optical fiber bundle 24 is 60% or more and 95% or less. In addition, the cross-sectional area of the first optical fiber bundle 24 is the area of a circle having the maximum external diameter of the optical fiber bundle as the diameter thereof. By setting the filling factor to 60% or more, longitudinal displacement of the first optical fiber bundle 24 inside the first protective tube 40 can be suppressed, and the deflection of the first optical fiber bundle 24 more than needed along the gaps between the first optical fiber bundle 24 and the other built-in objects can be effectively suppressed using the stiffness of the first protective tube 40. Additionally, by setting the filling factor to 95% or less, it is easy to insert the first optical fiber bundle 24 through the first protective tube 40.

Moreover, the first protective tube 40 covers the first optical fiber bundle 24 from the distal end part 14 to the operating part 11, and the second protective tube 41 and the third protective tube 43 having a bending stiffness smaller than the first protective tube 40 covers the first optical fiber bundle 24 from the proximal end part of the first protective tube 40 to the terminal of the universal cord 12. Accordingly, the flexibility of the universal cord 12 is enhanced, and the assemblibility and handlability of the endoscope 2 are enhanced.

Although the second protective tube 41 may extend from the proximal end part of the first protective tube 40 to the terminal of the universal cord 12, the second protective tube 41 covers the first optical fiber bundle 24 inside the operating part 11, and the third protective tube 43 having a bending stiffness smaller than the second protective tube 41 covers the first optical fiber bundle 24 from the proximal end part of the second protective tube 41 to the terminal of the universal cord 12. Accordingly, the flexibility of the universal cord 12 increases further. Accordingly, the assemblibility and handlability of the endoscope 2 are further enhanced. Additionally, the diameter of the universal cord 12 can also be reduced by integrally covering the first optical fiber bundle 24 and the second optical fiber bundle 25 with the third protective tube 43.

The contact between the built-in objects is likely to occur particularly around the first optical fiber bundle 24 in which the built-in objects are relatively densely disposed. Hence, it is particularly useful to cover the first optical fiber bundle 24 with the first protective tube 40 made of the non-porous (solid) fluororesin from the distal end part 14 to the operating part 11. Meanwhile, the contact between the built-in objects is reduced around the second optical fiber bundle 25 in which the built-in objects are relatively sparsely disposed. Hence, the configuration of the protective tube that covers the second optical fiber bundle 25 has a higher degree of freedom than the configuration of the protective tube that covers the first optical fiber bundle 24.

The light guide 20A illustrated in FIG. 4 further has a fourth protective tube 50 and a fifth protective tube 51 as protective tubes that cover the second optical fiber bundle 25.

The fourth protective tube 50 covers the second optical fiber bundle 25 from the distal end part 14 to the operating part 11 and is made of the non-porous (solid) fluororesin similarly to the first protective tube 40. The fifth protective tube 51 is connected to a proximal end part of the fourth protective tube 50 and covers the second optical fiber bundle 25. The fifth protective tube 51 is made of the porous fluororesin, the polyolefin, the silicone resin, or the like similarly to the second protective tube 41 and has a bending stiffness smaller than the fourth protective tube 50.

Since the fourth protective tube 50 made of the non-porous (solid) fluororesin have low friction and high stiffness and covers the second optical fiber bundle 25 from the distal end part 14 to the operating part 11, meandering of the second optical fiber bundle 25 can be suppressed, and breakage of an optical fiber resulting from the meandering can be suppressed. It is preferable that a filling factor that is a ratio between the inner cross-sectional area of the fourth protective tube 50 and the cross-sectional area of the second optical fiber bundle 25 is 60% or more and 95% or less.

Preferably, a connecting part 42 between the first protective tube 40 and the second protective tube 41 and a connecting part 52 between the fourth protective tube 50 and the fifth protective tube 51 is disposed so as to shift away from each other in the longitudinal direction of the light guide 20A. By disposing the connecting part 42 having larger diameter than the first protective tube 40 and the second protective tube 41 and the connecting part 52 larger diameter than the fourth protective tube 50 and the fifth protective tube 51 so as to shift away from each other, the density of the built-in objects inside the operating part 11 is relaxed, and any contact between built-in objects is reduced.

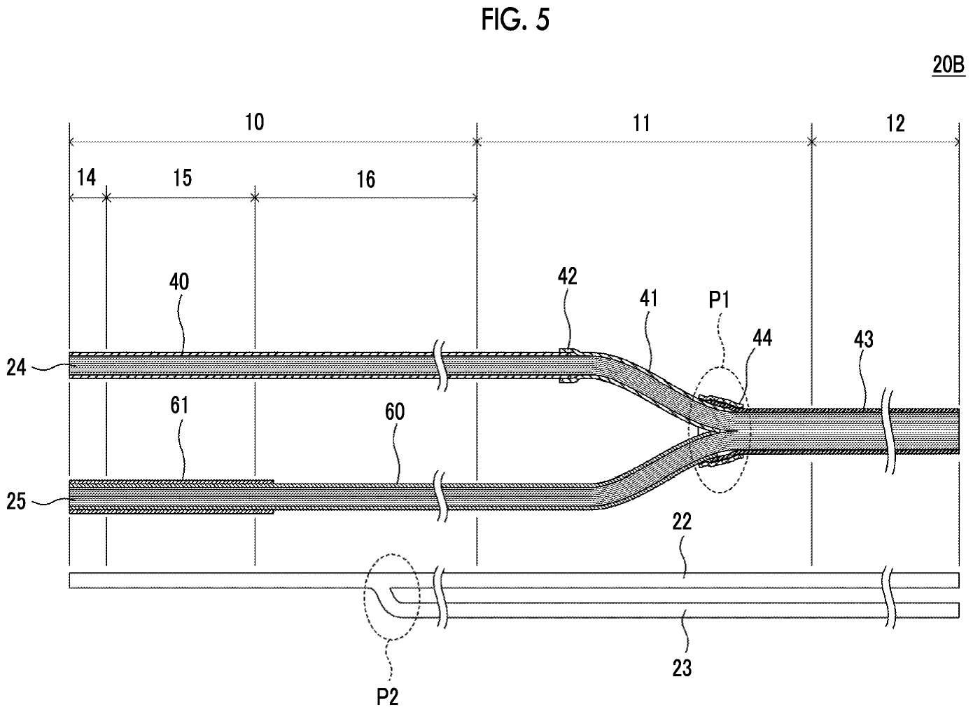

The light guide 20B illustrated in FIG. 5 further has a fourth protective tube 60 and a fifth protective tube 61 as protective tubes that cover the second optical fiber bundle 25.

The fourth protective tube 60 covers the second optical fiber bundle 25 from the distal end part 14 of the insertion part 10 to the joining point P1 between the first optical fiber bundle 24 and the second optical fiber bundle 25. The fourth protective tube 60 has a bending stiffness smaller than the first protective tube 40 made of the non-porous (solid) fluororesin. The silicone resin and the like can be exemplified as materials of the fourth protective tube 60. The fifth protective tube 61 covers the fourth protective tube 50 from the distal end part 14 to a portion of the flexible part 16 on the bending part 15 side. The fifth protective tube 61 made of the porous fluororesin.

In addition, similarly to the light guide 20A illustrated in FIG. 4, the third protective tube 43 integrally covers the first optical fiber bundle 24 and the second optical fiber bundle 25 from the joining point P1 between the first optical fiber bundle 24 and the second optical fiber bundle 25 to the terminal of the universal cord 12.

In the present example, in the bending part 15 in which the bending and bending-releasing operations are frequently performed, the second optical fiber bundle 25 is doubly covered with the fourth protective tube 60 and the fifth protective tube 61. Thus, the bending stiffness in the bending part 15 is enhanced. Accordingly, the meandering of the second optical fiber bundle 25 can be suppressed, and breakage of an optical fiber resulting from the meandering can be suppressed. In the flexible part 16, the second optical fiber bundle 25 is covered with the fourth protective tube 60. Thus, the fourth protective tube 60 has a bending stiffness smaller than the first protective tube 40 made of the non-porous (solid) fluororesin and has a bending stiffness smaller than the fourth protective tube 50 of the light guide 20A illustrated in FIG. 4. Accordingly, the flexibility of the flexible part 16 is enhanced, and the assemblibility and handlability of the endoscope 2 are enhanced.

Preferably, a proximal end part of the fifth protective tube 61 is disposed closer to the distal end part 14 side than a joining point P2 between the air supply pipe 22 and the water supply pipe 23. That is, a range where the second optical fiber bundle 25 is doubly covered with the fourth protective tube 60 and the fifth protective tube 61 is disposed in a section where the air supply pipe 22 and the water supply pipe 23 are joined together in the insertion part 10. Accordingly, high density of the built-in objects inside the insertion part 10 can be relaxed, and any contact between the built-in objects can be reduced.

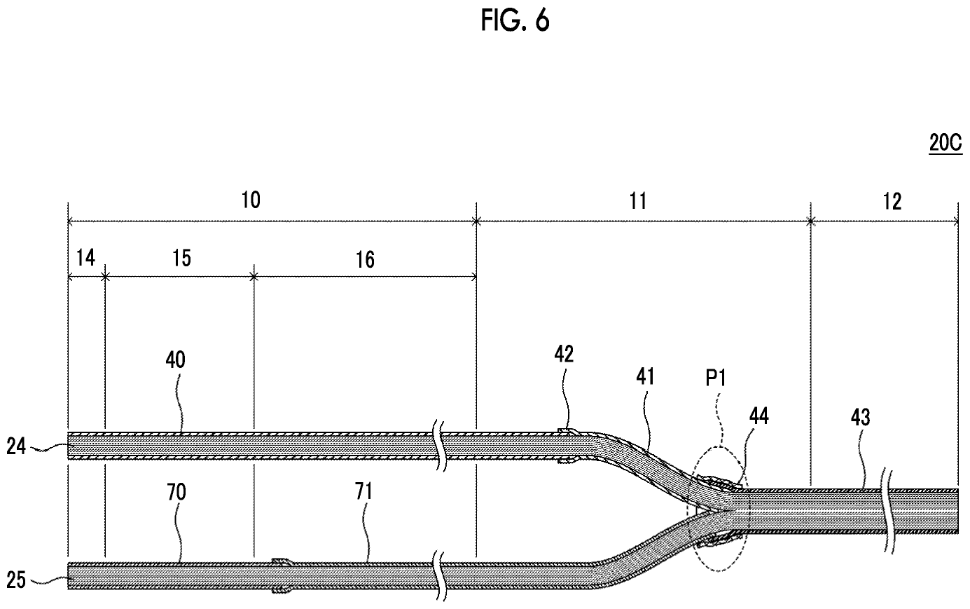

The light guide 20C illustrated in FIG. 6 further has a fourth protective tube 70 and a fifth protective tube 71 as protective tubes that cover the second optical fiber bundle 25.

The fourth protective tube 70 covers the second optical fiber bundle 25 from the distal end part 14 to a portion of the flexible part 16 on the bending part 15 side. The fourth protective tube 70 is made of the non-porous (solid) fluororesin. The fifth protective tube 71 covers the second optical fiber bundle 25 from a proximal end part of the fourth protective tube 70 to the joining point P1 between the first optical fiber bundle 24 and the second optical fiber bundle 25. The fifth protective tube 71 made of the porous fluororesin, the polyolefin, the silicone resin, or the like, and has a bending stiffness smaller than the fourth protective tube 70.

In addition, similarly to the light guide 20A illustrated in FIG. 4, the third protective tube 43 integrally covers the first optical fiber bundle 24 and the second optical fiber bundle 25 from the joining point P1 between the first optical fiber bundle 24 and the second optical fiber bundle 25 to the terminal of the universal cord 12.

In the present example, in the bending part 15 in which the bending and bending-releasing operations are frequently performed, the second optical fiber bundle 25 is doubly covered with the high-stiffness fourth protective tube 70 made of the non-porous (solid) fluororesin. Thus, the bending stiffness in the bending part 15 is enhanced. Accordingly, the meandering of the second optical fiber bundle 25 can be suppressed, and breakage of an optical fiber resulting from the meandering can be suppressed. Moreover, in the bending part 15, the second optical fiber bundle 25 is only covered with the fourth protective tube 70, and is also reduced in diameter compared to the case where the second optical fiber bundle 25 is doubly covered. Accordingly, high density of the built-in objects inside the bending part 15 can be relaxed, and any contact between the built-in objects can be reduced. In the flexible part 16, the second optical fiber bundle 25 is covered with the fifth protective tube 71. Thus, the fifth protective tube 71 has a bending stiffness smaller than the fourth protective tube 70 made of the non-porous (solid) fluororesin. Accordingly, the flexibility of the flexible part 16 is enhanced, and the assemblibility and handlability of the endoscope 2 are enhanced.

As described above, an endoscope disclosed in the present specification comprises a light guide that guides illumination light created by a light source device from the light source device via an operating part of the endoscope to a distal end part of an insertion part of the endoscope through an interior of an endoscope. The light guide includes a first optical fiber bundle; a first protective tube that covers the first optical fiber bundle from the distal end part of the insertion part to the operating part; and a second protective tube that is connected to an end part of the first protective tube on the light source device side and covers the first optical fiber bundle. The first protective tube is made of a non-porous (solid) fluororesin. The second protective tube has a bending stiffness smaller than the first protective tube.

Additionally, in the endoscope disclosed in the present specification, the second protective tube covers the first optical fiber bundle inside the operating part. The light guide has a third protective tube that is connected to an end part of the second protective tube on the light source device side and covers the first optical fiber bundle. The third protective tube has a bending stiffness smaller than the second protective tube.

Additionally, in the endoscope disclosed in the present specification, the light guide has a second optical fiber bundle that is separated from the first optical fiber bundle from the distal end part of the insertion part to the operating part and is joined to the first optical fiber bundle inside the operating part. The first optical fiber bundle is bundled with the second optical fiber bundle in a portion covered with the second protective tube. The third protective tube integrally covers the first optical fiber bundle and the second optical fiber bundle.

Additionally, in the endoscope disclosed in the present specification, the light guide has a fourth protective tube that covers the second optical fiber bundle from the distal end part of the insertion part to a joining point between the first optical fiber bundle and the second optical fiber bundle, and a fifth protective tube that covers the fourth protective tube from the distal end part of the insertion part via a bending part of the insertion part to a portion of a flexible part of the insertion part on the bending part side. The fourth protective tube has a bending stiffness smaller than the first protective tube. The fifth protective tube is made of a porous fluororesin.

Additionally, the endoscope disclosed in the present specification further comprises an air supply pipe that sends gas to the distal end part of the insertion part through the interior of the endoscope; and a water supply pipe that sends liquid to the distal end part of the insertion part through the interior of the endoscope. The air supply pipe and the water supply pipe are joined together in the flexible part of the insertion part and reach the distal end part of the insertion part. An end part of the fifth protective tube on the light source device side is disposed closer to the distal end part side of the insertion part than a joining point between the air supply pipe and the water supply pipe.

Additionally, in the endoscope disclosed in the present specification, the light guide has a fourth protective tube that covers the second optical fiber bundle from the distal end part of the insertion part to the operating part, and a fifth protective tube that is connected to an end part of the fourth protective tube on the light source device side and covers the second optical fiber bundle from the end part to the joining point between the first optical fiber bundle and the second optical fiber bundle. The fourth protective tube is made of the non-porous (solid) fluororesin. The fifth protective tube has a bending stiffness smaller than the fourth protective tube.

Additionally, in the endoscope disclosed in the present specification, a connecting part between the first protective tube and the second protective tube and a connecting part between the fourth protective tube and the fifth protective tube are disposed so as to shift away from each other in a longitudinal direction of the light guide.

Additionally, in the endoscope disclosed in the present specification, a filling factor that is a ratio between an inner cross-sectional area of the first protective tube and a cross-sectional area of the first optical fiber bundle is 60% or more and 95% or less.

Additionally, in the endoscope disclosed in the present specification, a filling factor that is a ratio between an inner cross-sectional area of the fourth protective tube and a cross-sectional area of the second optical fiber bundle is 60% or more and 95% or less.

### EXPLANATION OF REFERENCES

1: endoscope system

2: endoscope

3: light source device

4: processor

5: water supply tank

6: monitor

10: insertion part

11: operating part

12: universal cord

13: connector

14: distal end part

15: bending part

16: flexible part

17: imaging device

18: treatment tool insertion port

20, 20A, 20B, and 20C: light guide

21: electrical cable

22: air supply pipe

23: water supply pipe

24: first optical fiber bundle

25: second optical fiber bundle

26: first illumination window

27: second illumination window

28: observation window

29: mouthpiece

30: connecting pipe

31: nozzle

32, 32a, 32b, 32c, and 32d: operating wire

33: treatment tool channel

40: first protective tube

41: second protective tube

42: connecting part

43: third protective tube

44: binding member

50: fourth protective tube

51: fifth protective tube

52: connecting part

60: fourth protective tube

61: fifth protective tube

70: fourth protective tube

71: fifth protective tube

P1: joining point

P2: joining point

X: first axis

Y: second axis

What is claimed is:

1. An endoscope comprising:
a light guide that guides illumination light generated by a light source device from the light source device via an operating part of the endoscope to a distal end part of an insertion part of the endoscope through an interior of the endoscope,
wherein the light guide includes:
a first optical fiber bundle;
a first protective tube that covers the first optical fiber bundle from the distal end part of the insertion part to the operating part; and
a second protective tube that is connected to an end part of the first protective tube on a side of the light source device and covers the first optical fiber bundle,
wherein the first protective tube is made of a non-porous fluororesin, and
wherein the second protective tube has a bending stiffness smaller than a bending stiffness of the first protective tube.

2. The endoscope according to claim 1,
wherein the second protective tube covers the first optical fiber bundle inside the operating part,
wherein the light guide has a third protective tube that is connected to an end part of the second protective tube on a side of the light source device and covers the first optical fiber bundle, and
wherein the third protective tube has a bending stiffness smaller than the bending stiffness of the second protective tube.

3. The endoscope according to claim 2,
wherein the light guide has a second optical fiber bundle that is separated from the first optical fiber bundle from the distal end part of the insertion part to the operating part and joins the first optical fiber bundle inside the operating part,
wherein the first optical fiber bundle is bundled with the second optical fiber bundle in a portion covered with the second protective tube, and
wherein the third protective tube integrally covers the first optical fiber bundle and the second optical fiber bundle.

4. The endoscope according to claim 3,
wherein the light guide has a fourth protective tube that covers the second optical fiber bundle from the distal end part of the insertion part to a joining point of the first optical fiber bundle and the second optical fiber bundle, and a fifth protective tube that covers the fourth protective tube from the distal end part of the insertion part via a bending part of the insertion part to a portion of a flexible part of the insertion part on a side of the bending part,
wherein the fourth protective tube has a bending stiffness smaller than the bending stiffness of the first protective tube, and
wherein the fifth protective tube is made of a porous fluororesin.

5. The endoscope according to claim 4, further comprising:
an air supply pipe that sends gas to the distal end part of the insertion part through the interior of the endoscope; and
a water supply pipe that sends liquid to the distal end part of the insertion part through the interior of the endoscope,
wherein the air supply pipe and the water supply pipe join each other in the flexible part of the insertion part and reach the distal end part of the insertion part, and
wherein an end part of the fifth protective tube on a side of the light source device is disposed at a side of the distal end part of the insertion part with respect to a joining point between the air supply pipe and the water supply pipe.

6. The endoscope according to claim 3,
wherein the light guide has:
a fourth protective tube that covers the second optical fiber bundle from the distal end part of the insertion part to the operating part, and
a fifth protective tube that is connected to an end part of the fourth protective tube on a side of the light source device and covers the second optical fiber bundle from the end part to the joining point of the first optical fiber bundle and the second optical fiber bundle,
wherein the fourth protective tube is made of a non-porous fluororesin, and
wherein the fifth protective tube has a bending stiffness smaller than the bending stiffness of the fourth protective tube.

7. The endoscope according to claim 6,
wherein a connecting part between the first protective tube and the second protective tube and a connecting part between the fourth protective tube and the fifth protective tube are disposed so as to shift away from each other in a longitudinal direction of the light guide.

8. The endoscope according to claim 1,
wherein a filling factor that is a ratio between an inner cross-sectional area of the first protective tube and a cross-sectional area of the first optical fiber bundle is between 60% or more and 95% or less.

9. The endoscope according to claim 2,
wherein a filling factor that is a ratio between an inner cross-sectional area of the first protective tube and a cross-sectional area of the first optical fiber bundle is between 60% or more and 95% or less.

10. The endoscope according to claim 3,
wherein a filling factor that is a ratio between an inner cross-sectional area of the first protective tube and a cross-sectional area of the first optical fiber bundle is between 60% or more and 95% or less.

11. The endoscope according to claim 4,
wherein a filling factor that is a ratio between an inner cross-sectional area of the first protective tube and a cross-sectional area of the first optical fiber bundle is between 60% or more and 95% or less.

12. The endoscope according to claim 5,
wherein a filling factor that is a ratio between an inner cross-sectional area of the first protective tube and a cross-sectional area of the first optical fiber bundle is between 60% or more and 95% or less.

13. The endoscope according to claim 6,
wherein a filling factor that is a ratio between an inner cross-sectional area of the first protective tube and a cross-sectional area of the first optical fiber bundle is between 60% or more and 95% or less.

14. The endoscope according to claim 7,
wherein a filling factor that is a ratio between an inner cross-sectional area of the first protective tube and a cross-sectional area of the first optical fiber bundle is between 60% or more and 95% or less.

15. The endoscope according to claim 6,
wherein a filling factor that is a ratio between an inner cross-sectional area of the fourth protective tube and a cross-sectional area of the second optical fiber bundle is between 60% or more and 95% or less.

16. The endoscope according to claim 7,
wherein a filling factor that is a ratio between an inner cross-sectional area of the fourth protective tube and a cross-sectional area of the second optical fiber bundle is between 60% or more and 95% or less.
